(19) United States
(12) Patent Application Publication
(10) Pub. No.: US 2006/0029674 A1
(43) Pub. Date: Feb. 9, 2006

(54) Stable amorphous Cefdinir

(76) Inventors: Nancy E. Sever, Arlington Heights, IL (US); Devalina Law, Libertyville, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/103,183

(22) Filed: Apr. 11, 2005

Related U.S. Application Data
(60) Provisional application No. 60/560,957, filed on Apr. 9, 2004.

(51) Int. Cl. A61K 31/545; C07D 501/00; A61K 9/14

(52) U.S. Cl. 424/486; 514/202; 540/222

(57) ABSTRACT

The present invention relates to stable amorphous 7-[2-(2-aminothiazol-4-yl)-2-hydroxyiminoacetamide]-3-vinyl-3-cephem-4-carboxylic acid (syn isomer), methods for its preparation, and pharmaceutical compositions comprising stable amorphous 7-[2-(2-aminothiazol-4-yl)-2-hydroxyiminoacetamide]-3-vinyl-3-cephem-4-carboxylic acid (syn isomer).

This application claims priority from U.S. Provisional Patent Application Ser. No. 60/560,957, filed Apr. 9, 2004, incorporated herein by reference.

### TECHNICAL FIELD

The present invention relates to stable amorphous 7-[2-(2-aminothiazol-4-yl)-2-hydroxyiminoacetamide]-3-vinyl-3-cephem-4-carboxylic acid (syn isomer), formulations thereof, methods for their preparation, and pharmaceutical compositions comprising the stable amorphous compound.

### BACKGROUND OF THE INVENTION

The antimicrobial agent 7-[2-(2-aminothiazol-4-yl)-2-hydroxyiminoacetamido]-3-vinyl-3-cephem-4-carboxylic acid (syn isomer) (hereinafter referred to as “Cefdinir”) is a semi-synthetic oral antibiotic in the cephalosporin family. Cefdinir is sold in the United States as Omnicef® in capsule and oral suspension forms. Omnicef® is active against a wide spectrum of bacteria, including Staphylococcus aureus, Streptococcus pneumoniae, Streptococcus pogenes, Hemophilus influenzae, Moraxella catarrhalis, E. coli, Klebsiella, and Proteus mirabilis. The preparation of Cefdinir was first disclosed in U.S. Pat. No. 4,559,334, issued Dec. 17, 1985, while the preparation of the commercially available form of Cefdinir (Crystal A) was first disclosed in U.S. Pat. No. 4,935,507, issued Jun. 19, 1990, both of which are hereby incorporated by reference in their entirety.

The preparation of Cefdinir in U.S. Pat. No. 4,559,334 taught a crystalline-like amorphous material. However, the amorphous material was not pure and unstable.

The present invention provides a stable amorphous Cefdinir as well as formulations thereof, methods for their preparation, and pharmaceutical compositions and uses thereof. Pharmaceutical compositions comprising cefdinir are useful in treating bacterial infections such as Streptococcus pneumoniae and Hemophilus influenzae.

### BRIEF DESCRIPTION OF THE FIGURE

FIG. 1: X-ray diffraction pattern for Cefdinir monohydrate

FIG. 2: X-ray pattern of amorphous Cefdinir

FIG. 3: FTIR of amorphous Cefdinir

FIG. 4: TGA thermogram of amorphous Cefdinir during an isothermal hold at 25° C.

FIG. 5: Molecular structure of Eudragit EPO monomer

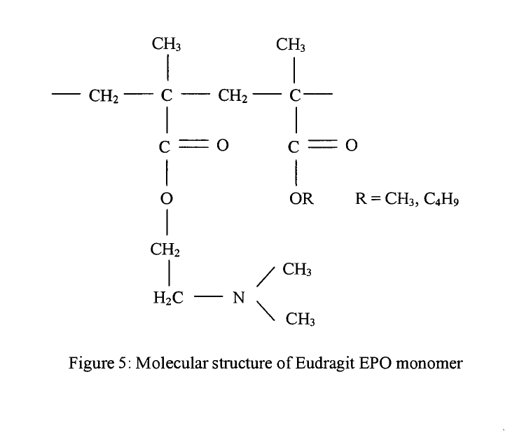

FIG. 6: X-ray pattern of amorphous Cefdinir with Eudragit EPO

FIG. 7a: Fit of Cefdinir/EPO spectra using deconvolution peaks from the pure components

FIG. 7b: Fit of Cefdinir/EPO spectra using an additional peak at 1612 cm⁻¹

FIG. 8: TGA thermogram of amorphous Cefdinir in Eudragit EPO during an isothermal hold at 25° C.

FIG. 9: Molecular structure of PVP

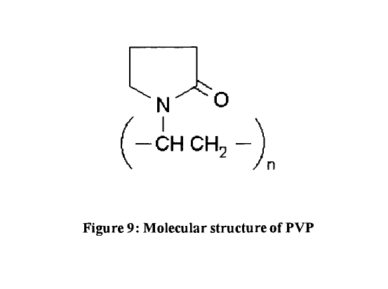

FIG. 10: FT-IR spectrum of amorphous Cefdinir/PVP, amorphous Cefdinir and PVP

FIG. 11: TGA thermogram amorphous Cefdinir in PVP during an isothermal hold at 25° C.

### SUMMARY OF THE INVENTION

The present invention relates to stable amorphous 7-[2-(2-aminothiazol-4-yl)-2-hydroxyiminoacetamide]-3-vinyl-3-cephem-4-carboxylic acid (syn isomer), methods for its preparation, and pharmaceutical compositions comprising stable amorphous 7-[2-(2-aminothiazol-4-yl)-2-hydroxyiminoacetamide]-3-vinyl-3-cephem-4-carboxylic acid (syn isomer).

### DETAILED DESCRIPTION OF THE INVENTION

The present invention relates to stable amorphous 7-[2-(2-aminothiazol-4-yl)-2-hydroxyiminoacetamide]-3-vinyl-3-cephem-4-carboxylic acid (syn isomer), methods for its preparation, and pharmaceutical compositions comprising stable amorphous 7-[2-(2-aminothiazol-4-yl)-2-hydroxyiminoacetamide]-3-vinyl-3-cephem-4-carboxylic acid (syn isomer).

The present invention also relates to making cefdinir (Crystal A) from amorphous cefdinir by combining amorphous cefdinir in a solvent, such as, but not limited to, water.

The present invention also relates to stable amorphous 7-[2-(2-aminothiazol-4-yl)-2-hydroxyiminoacetamide]-3-vinyl-3-cephem-4-carboxylic acid (syn isomer) that is combined with any cationic polymer. The present invention also relates to stable amorphous 7-[2-(2-aminothiazol-4-yl)-2-hydroxyiminoacetamide]-3-vinyl-3-cephem-4-carboxylic acid (syn isomer) that is combined with any amorphous neutral polymer or copolymer. The present invention also relates to stable amorphous 7-[2-(2-aminothiazol-4-yl)-2-hydroxyiminoacetamide]-3-vinyl-3-cephem-4-carboxylic acid (syn isomer) that is combined with any amorphous cationic polymer with an acid dissociation constant greater than 2.

Stable amorphous cefdinir can also be made with cationic polymers. In particular, stable amorphous cefdinir can be combined with a amorphous cationic polymer with an acid dissociation constant greater than 2. Suitable cationic polymers include, but are not limited to, Eudragit E series of polymers.

Stable amorphous cefdinir can also be made with neutral polymers or copolymers. Suitable neutral polymers or copolymers include, but are not limited to, PVPs, PVAs, PVP-co-PVA (copovidon), HEC, HPMC, HPMCP (hydroxypropyl methylcellulose phthalate). Amorphous cefdinir with PVP was made and isolated by evaporating a methanolic solution. The amorphous material was physically stable.

Stable amorphous cefdinir can also be made with anionic polymers. Suitable anionic polymers include, but are not limited to, Eudragit L series of polymers and carbapols.

Stable amorphous cefdinir can also be made with macromolecules. Suitable macromolecules include, but are not limited to, dextrin (dextrose polymer) and maltodextrin.

The present invention also relates to stable amorphous 7-[2-(2-aminothiazol-4-yl)-2-hydroxyiminoacetamide]-3-vinyl-3-cephem-4-carboxylic acid (syn isomer) that is combined with any amorphous polymer. The present invention also relates to stable amorphous 7-[2-(2-aminothiazol-4-yl)-2-hydroxyiminoacetamide]-3-vinyl-3-cephem-4-carboxylic acid (syn isomer) that is combined with polyvinylpyrollidone or any other amorphous polymer such as HPMCs.

The present invention also relates to stable amorphous 7-[2-(2-aminothiazol-4-yl)-2-hydroxyiminoacetamide]-3-vinyl-3-cephem-4-carboxylic acid (syn isomer) that is prepared by combining a cefdinir hydrate in an organic solvent and then evaporating the solution.

Powder X-ray diffraction (PXRD) was performed using an XDS-2000/X-ray diffractometer equipped with a 2 kW normal focus X-ray tube and a Peltier cooled germanium solid-state detector (Scintag Inc., Sunnyvale, Calif.). The data was processed using DMSNT software (version 1.37). The X-ray source was a copper filament operated at 45 kV and 40 mA. The alignment of the goniometer was checked daily using a Corundum standard. The sample was placed in a thin layer onto a zero background plate, and continuously scanned at a rate of 2° two-theta per minute over a range of 2 to 40° two-theta.

Characteristic powder X-ray diffraction pattern peak positions are reported in terms of the angular positions (two theta) with an allowable variability of ±0.1°. This allowable variability is specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (two theta) which is the measured peak position ±0.1° and if those ranges of peak positions overlap, then the two peaks are considered to have the same angular position (two theta). For example, if a diffraction pattern peak from one pattern is determined to have a peak position of 5.2°, for comparison purposes the allowable variability allows the peak to be assigned a position in the range of 5.1°-5.3°. If a comparison peak from the other diffraction pattern is determined to have a peak position of 5.3°, for comparison purposes the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position (two theta).

Transmission infrared spectra of the solids were obtained using a Fourier-transform infrared spectrometer (FTIR) (Nicolet Magna 750 FT-IR Spectrometer, Nicolet Instrument Corporation, Madison, Wis.) equipped with a Nicolet NIC-PLAN microscope. The microscope had an MCT-A liquid nitrogen cooled detector. The samples were rolled on a 13 mm×1 mm BaF₂ disc sample holder, 64 scans were collected at 4 cm⁻¹ resolution.

Thermogravimetric analysis (TGA) was performed in TA Instruments TG2950 (TA Instruments, New Castle, Del.). The samples were scanned at 10° C./minute with a dry nitrogen purge at 60 mL/minute.

Briefly, the process for the preparation of cefdinir is detailed below.

To a solution of benzhydryl 7-(4-bromoacetoacetamido)-3-vinyl-3-cephem-4-carboxylate (10 g) in a mixture of methylene chloride (70 ml) and acetic acid (25 ml) is dropwise added isoamylnitrite (3.5 ml) at −3° to −5° C. The mixture is stirred for 40 minutes at −5° C., followed by addition of acetylacetone (4 g) and stirring for 30 minutes at 5° C. To the reaction mixture is added thiourea (3 g) and stirring for 3 hours, then added dropwise is ethyl acetate (70 ml) and diisopropyl ether (100 ml). The resultant precipitate is collected by filtration and dried in vacuo to give benzhydryl 7-[2-(-aminothiazaol-4-yl)-2-hydroxyiminoacetamido]-3-vinyl-3-cephem-4-carboxylate hydrobromide (syn isomer) This product is added portionwise to a mixture of 2,2,2-trifluroacetic acid and anisole at 5° to 7° C. After stirring for 1 hour at 5° C., the reaction mixture is added dropwise to diisopropyl ether (150 ml). The resultant precipitate is collected by filtration and dissolved in a mixture of terahydrofuran (10 ml) and ethyl acetate (10 ml). The organic layer is extracted with aqueous sodium bicarbonate. The aqueous extract is washed with ethyl acetate while keeping the pH value at 5 and then adjusted to pH 2.2 with 10% hydrochloric acid. This solution is stirred for 1 hour at 0° C., and the obtained crystals collected by filtration and dried in vacuo to give 7-[2-(2-aminothiazol-4-yl)-2-hydroxyiminoacetamido]-3-vinyl-3 cephem-4-carboxylic acid (syn isomer).

Alternatively, to a solution of benzhydryl 7-[2-(2-aminothiazol-4-yl)-2-hydroxyiminoacetamido]-3-vinyl-3-cephem-4-carboxylate (syn isomer) (5 g) in a mixture of anisole (20 ml) and acetic acid (5 ml) is added dropwise boron trufuloride etherate (5 ml) at 10° C. After stirring for 20 minutes at 10° C., the reaction mixture is poured into a mixture of tetrahydrofuran (100 ml), ethyl acetate (100 ml) and water (100 ml), and then adjusted to pH 6.0 with 20% aqueous sodium hydroxide. The resultant aqueous layer is separated and washed with ethyl acetate under keeping pH value at 6.0. This solution is subjected to chromatography on aluminum oxide.

The fractions are eluted with 3% aqueous sodium acetate and are collected and adjusted to pH 4.0 with 10% hydrochloric acid. This solution is further chromatographed on nonionic absorption resin “Diaion HP-20” (Trademark, manufactured by Mitsubishi Chemical Industries). The fractions are eluted with 20% aqueous acetone and collected, concentrated in vacuo and adjusted to pH 2.0 with 10% hydrochloric acid. The resultant precipitate is collected by filtration and dried in vacuo to give 7-[2-(2-aminotiazol-4-yl)-2-hydroxyminioacetamido]-3-vinyl-3-cephem-4-carboxylic acid (syn isomer). Further purification procedures can be performed to provide a suitable product.

Crystal A of Cefdinir

A pure cefdinir can be obtained by acidifying the solution containing cefdinir at room temperature or under warming and thereby having the crystals separate out of the solution.

Suitable examples of the solution containing cefdinir may include, for example, an aqueous solution of the alkali metal salt of cefdinir. The solution containing cefdinir is acidified, if necessary, after said solution is subjected to a column chromatography on activated charcoal, nonionic adsorption resin, alumina, acidic aluminium oxide. The acidifying process can be carried out by adding an acid such as hydrochloric acid or the like preferably in the temperature range from room temperature to 40° C., more preferably, from 15° to 40° C. The amount of the acid to be added preferably makes the pH value of the solution from about 1 to about 4.

A pure cefdinir can be also obtained by dissolving the cefdinir in an alcohol (preferably methanol), continuing to stir this solution slowly under warming (preferably below 40° C.), preferably after the addition of water warmed at almost the same temperature as that of said solution, then cooling this solution to room temperature and allowing it to stand.

During the crystallization of cefdinir, it is preferable to keep the amount slightly beyond the saturation. Cefdinir obtained according to aforesaid process can be collected by filtration and dried by means of the conventional methods.

7-[2-(2-Aminothiazol-4-yl)-2-hydroxyminoacetamido]-3-vinyl-3-cephem-4-carboxylic acid (syn isomer) (29.55 g) can be added to water (300 ml) and the mixture adjusted to pH 6.0 with saturated sodium bicarbonate aqueous solution. The resultant solution can be subjected to a column chromatography on activated charcoal and eluted with 20% aqueous acetone. The fractions are combined and concentrated to a volume of 500 ml. The resultant solution pH is adjusted to 1.8 at 35° C. with 4N hydrochloric acid. The resultant precipitates are collected by filtration, washed with water and dried to give 7-[2-(2 aminothiazol-4-yl)-2-hydroxyminoacetamido]-3-vinyl-3-cephem-4-carboxylic acid (syn isomer).

Alternatively, to a solution of 7-[2-(2-aminothiazol-4-yl)-2-hydroxyminoacetamido]-3-vinyl-3-cephem-4-carboxylic acid (syn isomer) (0.5 g) in methanol (10 ml) can be added dropwise warm water (35° C.; 1.5 ml) at 35° C. and the resultant solution stirred slowly for 3 minutes, then allowed to stand at room temperature. The resultant crystals are collected by filtration, washed with water and then dried to give 7-[2(2-3-aminothiazol-4-yl)-2-hydroxyminioacetamido]3-vinyl-3-cephem-4-carboxylic acid (syn isomer) as crystals.

Cefdinir Hydrate

One method for preparing Cefdinir Hydrate involves: Cefdinir, ca. 0.1 g was suspended in 2 mL of a 1:1 ethanol: ethylacetate solution. To this suspension, approximately 2 drops of concentrated H₂SO₄ were added with intermittent sonication to obtain a clear solution. The solution was partially concentrated by evaporation and then carefully diluted with 60 mL water (or large excess of water). This clear solution was allowed to stand. Crystal growth was observed within an hour. The crystals isolated from this solution can be used or the crystals may be dried either at room temperature or 75° C. and the dried crystals may be used for preparing amorphous cefdinir.

Amorphous Cefdinir

Amorphous Cefdinir was isolated by evaporating a methanolic solution of cefdinir hydrate. The amorphous material was physically stable.

In a round bottom flask, 2 ml of methanol (HPLC Grade) and 0.05 g Cefdinir monohydrate were combined. The solution was mixed (vortexed and sonicated) until clear. House air was used to evaporate the solvent and dry the contents of the flask. The resultant product was a grainy powder at the bottom of the flask.

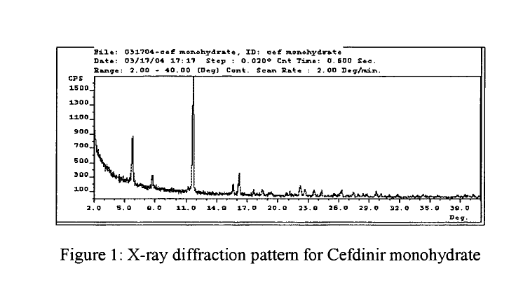

The powder x-ray diffraction pattern (2° to 40° at 2′/min) for the Cefdinir Monohydrate is shown in FIG. 1.

The powder isolated above was examined by microscopy and PXRD. Microscopy analysis, with a microscope equipped with cross polars, revealed that the particles appeared glassy and did not exhibit birefringence.

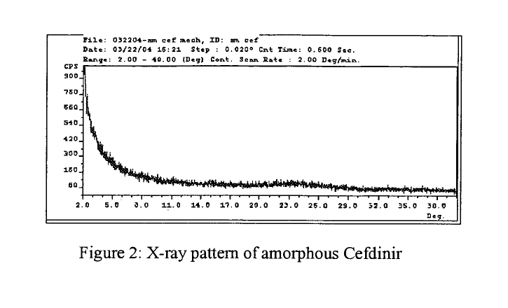

For the powder X-ray diffraction pattern, the sample was scanned from 2° to 40° at a rate of 20/min. The x-ray pattern lacked the characteristic crystalline peaks and showed the halo consistent with amorphous material (FIG. 2).

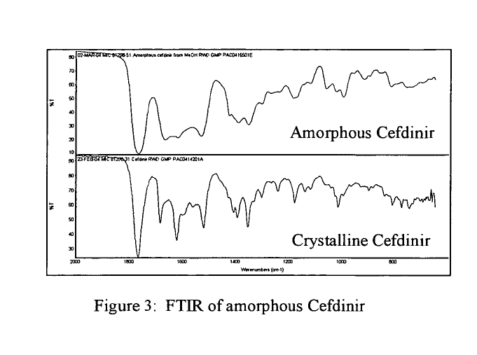

The FT-IR spectrum is an average of 64 scans at 4 cm⁻¹ resolution. FIG. 3 compares the spectra of the crystalline and amorphous Cefdinir powders. The spectrum showed peaks at locations consistent with the crystalline material indicating that the amorphous material is chemically similar to crystalline Cefdinir. As expected, the peaks in the amorphous material were less sharp.

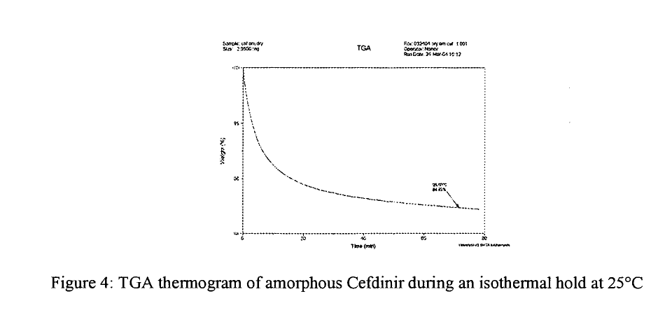

The residual solvent can be removed by holding the sample in the TGA for 1 hour at 25° C. (FIG. 4). At the end of the hour, the weight reached a constant value and the sample had lost 5% of its weight. From this data it was concluded that the amorphous material had 5% residual solvent.

For High Pressure Liquid Chromatography (HPLC), the sample was isolated by evaporating methanol and analyzed by HPLC for potency. After accounting of the 5 wt % residual solvent, the amorphous material obtained had a potency of 98%.

The glass transition temperature (T_{g}) determined by thermally stimulated current spectroscopy was 67° C. This value of 67° C. is considerably higher than ambient temperature, and as a rule of thumb high T_{g} values are desirable for room temperature stability.

Amorphous Cefdinir with Eudragit EPO

Stable amorphous cefdinir can also be made with cationic polymers. In particular, stable amorphous cefdinir can be combined with a amorphous cationic polymer with an acid dissociation constant greater than 2. Suitable cationic polymers include, but are not limited to, Eudragit E series of polymers.

Stable amorphous Cefdinir with Eudragit EPO was made and isolated by evaporating a methanolic solution. The amorphous material was physically stable.

In a round bottom flask, 0.05 g of Cefdinir monohydrate and 2 ml of HPLC grade methanol were combined. The solution was mixed (vortexed and sonicated) in a round bottom flask until clear. A 1:1 molar ratio of Eudragit EPO to Cefdinir was added. Eudragit EPO (0.036 g) was first dissolved in 0.5 ml of methanol, then added to the Cefdinir solution. Immediately upon the addition of Eudragit EPO, a white precipitate formed. Methanol was evaporated and the resultant product was a white film on the surface of the flask. The film was analyzed.

Characterization of Amorphous Cefdinir with Eudragit EPO

The powder isolated above was examined by microscopy and PXRD. Microscopy analysis, with a microscope equipped with cross polars, revealed that the particles appeared glassy and did not exhibit birefringence.

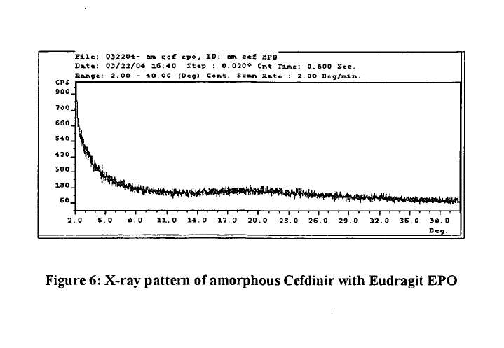

For the powder X-ray diffraction pattern, the sample was scanned from 2° to 40° at a rate of 2′/min. The x-ray pattern lacked the characteristic crystalline peaks and showed the halo consistent with amorphous material (FIG. 6).

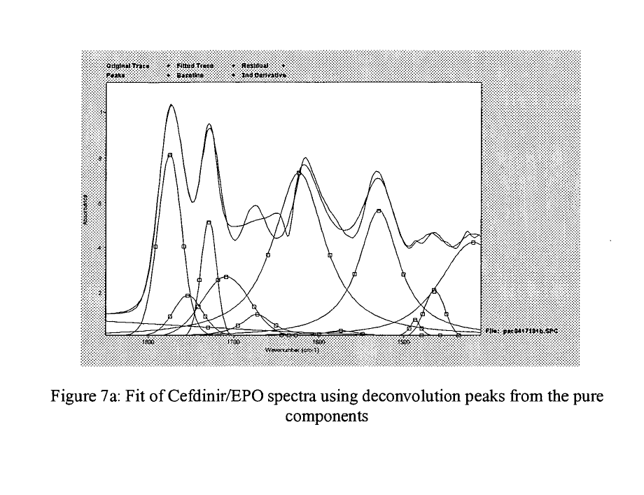

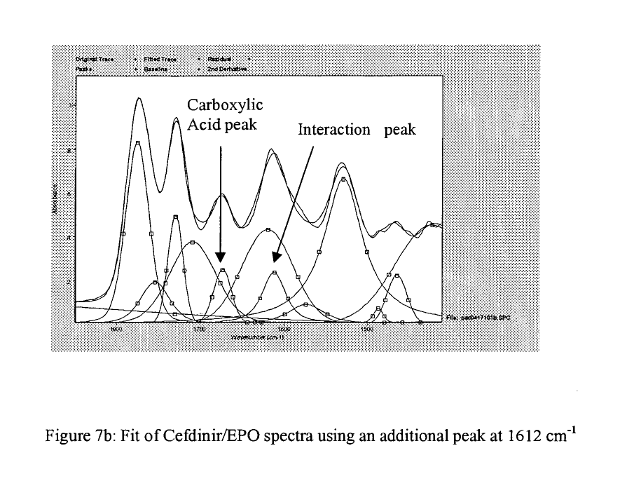

For the FT-IR spectrum, the spectrum is an average of 64 scans at 4 cm⁻¹ resolution. The cefdinir-Eudragit EPO spectrum appeared different from either amorphous cefdinir or Eudragit EPO, therefore the peaks of this spectrum were deconvoluted (FIG. 7a). The resultant spectrum had features that were not sufficient to fit the mixture spectrum. An additional peak was needed at 1612 cm⁻¹ to improve the fit as shown in FIG. 7b. The location of the additional peak is consistent with a salt formation. Therefore, analysis of the FT-IR data does support the formation of a complex between Eudragit EPO and cefdinir. Such specific interaction is expected to provide enhanced stability to the amorphous phase.

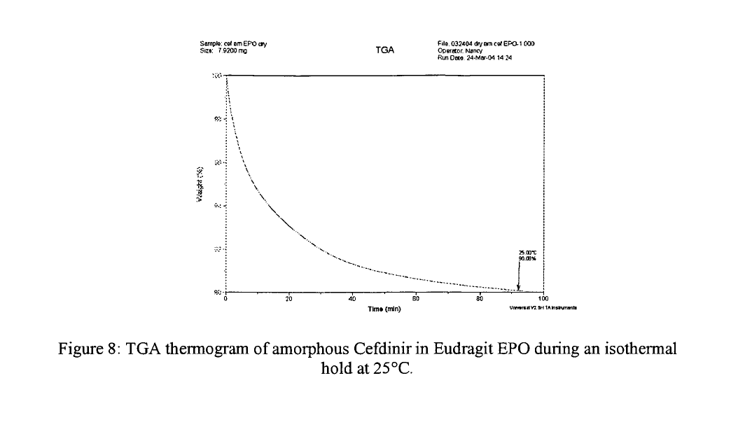

The residual methanol can be removed by holding the sample in the TGA for 1 hour at 25° C. (FIG. 8). At the end of the hour, the weight reached a constant value and the sample had lost 10% of its weight. From this data it was concluded that the amorphous material had 10% residual solvent.

For the HPLC analysis, the sample isolated by evaporating methanol was analyzed by HPLC for potency. After accounting of the 10 wt % residual solvent, the amorphous material obtained had a potency of about 99%.

The glass transition temperature (T_{g}) determined by thermally stimulated current spectroscopy was 102° C. Interestingly the T_{g} of amorphous cefdinir and Eudragit-EPO are 67° C. and 84° C., respectively but that of the dispersion containing the two components is higher (102° C.). The higher T_{g} observed for the cefdinir-EPO sample relative to the individual components further confirms specific interaction.

Amorphous Cefdinir with PVP

Stable amorphous cefdinir can also be made with neutral polymers or copolymers. Suitable neutral polymers or copolymers include, but are not limited to, PVPS, PVAs, PVP-co-PVA (copovidon), HEC, HPMC, HPMCP (hydroxypropyl methylcellulose phthalate).

Amorphous cefdinir with PVP was made and isolated by evaporating a methanolic solution. The amorphous material was physically stable.

In a round bottom flask, 2 ml of methanol (HPLC grade) and 0.05 g of Cefdinir monohydrate were combined. The solution was mixed (vortexed and sonicated) until clear. 80:20 w/w Polyvinylpyrrolidone K15 (PVP) to Cefdinir was added. The 0.2 g of PVP was first dissolved in 0.2 g of methanol, and then added to the Cefdinir solution. The solution remained clear. House air was used to evaporate the methanol and dry the contents of the flask. The resultant product was a clear film on the surface of the flask. The film was scraped off with a spatula.

Characterization of Amorphous Cefdinir with PVP

The isolated precipitate above was examined by microscopy and PXRD. Microscopy analysis, with a microscope equipped with cross polars, revealed that the particles appeared glassy and exhibited no birefringence.

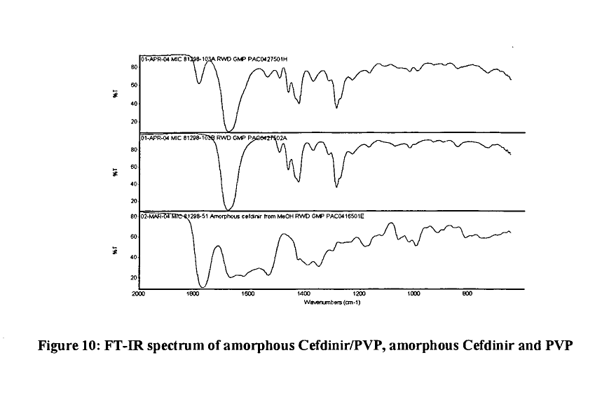

For the FT-IR analysis, the spectrum is an average of 64 scans at 4 cm⁻¹ resolution. A comparison of the crystalline Cefdinir and the amorphous Cefdinir/PVP sample is shown in FIG. 10. The spectra are similar and confirm the presence of Cefdinir in the amorphous material. The Cefdinir/PVP powder showed peaks at locations consistent with both the Amorphous Cefdinir and PVP. Due to the large amount of PVP present (80 wt %), the spectrum of the amorphous Cefdinir/PVP is more similar to that of PVP.

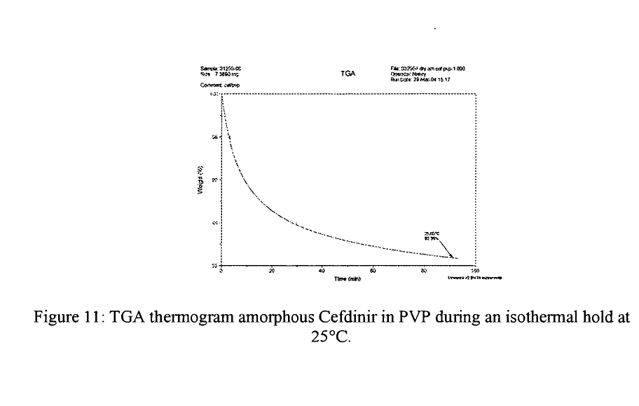

The residual methanol can be removed by holding the sample in the TGA for 1 hour at 25° C. (FIG. 11). At the end of the hour, the weight reached a constant value and the sample had lost 7% of its weight. From this data it was concluded that the amorphous material had 7% residual solvent.

The glass transition temperature (T_{g}) determined by thermally stimulated current spectroscopy was 95° C.

The process for preparation of stable amorphous cefdinir is critical. The use of the combination of a cefdinir hydrate and methanol allows rapid dissolution rate and avoids chemical degradation. The solvent is also good for the polymer and therefore one can start with a clear solution thus maximizing the chances of isolating the amorphous.

In accordance with methods of treatment and pharmaceutical compositions of the invention, the compounds can be administered alone or in combination with other agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, intranasally, rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The effect of parenterally administered compounds can be prolonged by slowing their release rates. One way to slow the release rate of a particular compound is administering injectable depot forms comprising suspensions of poorly soluble crystalline or otherwise water-insoluble forms of the compound. The release rate of the compound is dependent on its dissolution rate, which in turn, is dependent on its physical state. Another way to slow the release rate of a particular compound is administering injectable depot forms comprising the compound as an oleaginous solution or suspension. Yet another way to slow the release rate of a particular compound is administering injectable depot forms comprising microcapsule matrices of the compound trapped within liposomes, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of release can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise excipients such as sucrose, lactose, starch, microcrystalline cellulose, mannitol, talc, silicon dioxide, polyvinylpyrrolidone, sodium starch glycolate, magnesium stearate, etc. Capsules, tablets and pills can also comprise buffering agents, and tablets and pills can be prepared with enteric coatings or other release-controlling coatings. Powders and sprays can also contain excipients such as talc, silicon dioxide, sucrose, lactose, starch, or mixtures thereof. Sprays can additionally contain customary propellants such as chlorofluorohydrocarbons or substitutes thereof.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents. Liquid dosage forms may also be contained within soft elastic capsules.

Topical dosage forms include ointments, pastes, creams, lotions, gels, powders, solutions, sprays, inhalants, and transdermal patches. The compound is mixed, if necessary under sterile conditions, with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

Compositions comprising amorphous cefdinir are within the scope of this invention. In additon, formulations comprising the amorphous material with polymers such as, but not limited to, PVP and Eudragit, as well as methods of preparing stable amorphous cefdinir and formulations thereof are also within the scope of the present invention.

The foregoing is merely illustrative of the invention and is not intended to limit the invention to the disclosed embodiments. Variations and changes which are obvious to one skilled in the art are intended to be within the scope and nature of the invention which are defined in the appended claims.

What is claimed is:

1. Stable amorphous 7-[2-(2-aminothiazol-4-yl)-2-hydroxyiminoacetamido]-3-vinyl-3-cephem-4-carboxylic acid (syn isomer).

2. A pharmaceutical composition comprising stable amorphous 7-[2-(2-aminothiazol-4-yl)-2-hydroxyiminoacetamido]-3-vinyl-3-cephem-4-carboxylic acid (syn isomer).

3. A method of treating bacterial infections in a mammal using a pharmaceutical composition of claim 2.

4. A pharmaceutical composition comprising compound of claim 1 wherein the stable amorphous cefdinir is combined with a polymer or copolymer.

5. A pharmaceutical composition comprising compound of claim 1 wherein the stable amorphous cefdinir is combined with a amorphous cationic polymer.

6. A pharmaceutical composition of claim 5 wherein the cationic polymer has an acid dissociation constant greater than 2.

7. A pharmaceutical composition of claim 5 comprising the polymer Eudragit.

8. A pharmaceutical composition comprising compound of claim 1 wherein the stable amorphous cefdinir is combined with an amorphous polymer, copolymer or macromolecule.

9. A pharmacetical composition comprising the compound of claim 1 in composition with a neutral polymers or copolymer.

10. A pharmacetical composition of claim 9 wherein said neutral polymer or copolymer is selected from group consisting of PVPs, PVAS, PVP-co-PVA(copovidon), HEC (hydroxypropyl cellulose), HPMC, and HPMCP.

11. A pharmacetical composition comprising the compound of claim 1 in composition with a anionic polymer.

12. A pharmacetical composition of claim 11 wherein said anionic polymer is selected from the group consisting of eudragit Ls series of polymers and carbapols.

13. A pharmacetical composition comprising the compound of claim 1 in composition with a macromolecule.

14. A pharmacetical composition of claim 13 wherein said macromolecules is selected from dextrin and maltodextrin.

15. A process for producing stable amorphous cefdinir comprising combining a cefdinir hydrate in a methanolic solution and evaporating the solution.

16. A process for producing stable amorphous cefdinir comprising combining cefdinir monohydrate in an organic solvent in which the solubility of cefdinir monohydarte is greater than 0.5 mg/ml and evaoporating the solution.

17. A process for producing cefdinir Crystal A comprising combining amorphous cefdinir in a solvent.

18. A process for producing cefdinir Crystal A of claim 17 wherein said solvent is water.
